(19) United States
(12) Patent Application Publication
(10) Pub. No.: US 2013/0251683 A1
(43) Pub. Date: Sep. 26, 2013

(54) PROLONGED DELIVERY OF HEPARIN-BINDING GROWTH FACTORS FROM HEPARIN-DERIVATIZED COLLAGEN

(71) Applicant: GENZYME CORPORATION, Cambridge, MA (US)

(72) Inventors: Michael Santos, Cambridge, MA (US); Michael Philbrook, Boston, MA (US); Michael A. DiMicco, Brookline, MA (US); Robert J. Miller, East Bridgewater, MA (US)

(73) Assignee: GENZYME CORPORATION, Cambridge, MA (US)

(21) Appl. No.: 13/762,225

(22) Filed: Feb. 7, 2013

Related U.S. Application Data
(63) Continuation of application No. 12/248,307, filed on Oct. 9, 2008.
(60) Provisional application No. 60/998,118, filed on Oct. 9, 2007.

(51) Int. Cl. A61K 9/00; A61K 47/42

(52) U.S. Cl. CPC A61K 9/0002; A61K 47/42
USPC 424/93.6; 514/8.8

(57) ABSTRACT

The present invention relates to a heparin-derivatized collagen matrix comprising a fragment of heparin covalently linked to a collagen scaffold, wherein the fragment of heparin has molecular weight of less than about 15 kDa, and at least one heparin-binding growth factor (HBGF) or heparin-binding adeno-associated virus (HB-AAV) or a combination thereof and methods for promoting bone growth, bone repair, cartilage repair, bone development, neo-angiogensis, wound healing, tissue engraftment and muscle tissue regeneration and/or tissue augmentation comprising administering a heparin-derivatized collagen matrix that includes at least one heparin-binding growth factor or heparin-binding adeno-associated virus or a combination thereof.

### RELATED APPLICATION

This application is a continuation of U.S. application Ser. No. 12/248,307 filed Oct. 9, 2008, which claims the benefit of U.S. Provisional Application No. 60/998,118, filed on Oct. 9, 2007.

The entire teachings of the above application(s) are incorporated herein by reference.

### BACKGROUND OF THE INVENTION

Heparin-binding growth factors (HBGFs) and heparin-binding adeno-associated virus particles (HB-AAVs) can be useful as therapeutic agents to augment normal or impaired growth processes involving tissues in certain clinical states (e.g., wound healing). While therapeutic administration of exogenous HBGFs and/or HB-AAVs to sites of tissue injury has been used to control or modulate tissue growth, local delivery is complicated by the fact that growth factors show relatively short in vivo half lives due to proteolytic degradation and diffusion away from the injury site.

### SUMMARY OF THE INVENTION

The present invention relates to a heparin-derivatized collagen matrix comprising a fragment of heparin covalently linked to a collagen scaffold, wherein the fragment of heparin has a molecular weight of less that about 15 kDa, and at least one heparin-binding growth factor (HBGF) or heparin-binding adeno-associated virus particles (HB-AAVs) or combination thereof.

Examples of HBGFs of the present invention include, but are not limited to fibroblast growth factor (FGF), vascular endothelial growth factor (VEGF), heparin-binding epidermal growth factor (HBEGF), the transforming growth factor-b (TGF-β) superfamily, keratinocyte growth factor (KGF), pleiotrophin, placental growth factor (PlGF), hepatocyte growth factor, interferon-gamma (IFN-gamma), platelet-derived growth factor (PDGF), interleukin-8 (IL-8), macrophage inflammatory protein-1 (MIP-1), interferon-gamma-inducible protein-10 (IP-10) or HIV-Tat transactivating factor.

In a preferred embodiment of the invention, the HBGF is a member of the TGF-β superfamily. In another preferred embodiment, the member of the TGF-β superfamily is bone morphogenetic protein 2 (BMP-2). Both the TGF-β superfamily and bone morphogenetic protein 2 are known to play a role in bone and cartilage regeneration and repair.

In embodiments of the invention, the HB-AAV is selected from the group consisting of AAVs that promote the expression of fibroblast growth factor (FGF), vascular endothelial growth factor (VEGF), heparin-binding epidermal growth factor (HBEGF), the transforming growth factor-b (TGF-β) superfamily, keratinocyte growth factor (KGF), pleiotrophin, placental growth factor (PlGF), hepatocyte growth factor, interferon-gamma (IFN-gamma), platelet-derived growth factor (PDGF), interleukin-8 (IL-8), macrophage inflammatory protein-1 (MIP-1), interferon-gamma-inducible protein-10 (IP-10), HIV-Tat transactivating factor or combinations thereof.

In a preferred embodiment, the HB-AAV is adeno-associated virus-2 (AAV-2). In another preferred embodiment, the AAV-2 promotes the expression of one of the members of the TGF-β superfamily. In yet another preferred embodiment, the AAV-2 promotes the expression of BMP-2.

In embodiments of the invention, the fragment of heparin has a molecular weight of less than about 15 kDa. In a preferred embodiment, the fragment of heparin has a molecular weight between about 5 kDa and 13 kDa. In another preferred embodiment, the heparin fragment has a molecular weight between about 12 kDa and 13 kDa. In yet another preferred embodiment, the fragment of heparin has a molecular weight between about 5 kDa and 6 kDa.

The present invention also relates to methods for promoting bone growth, bone repair, bone development, cartilage repair, neo-angiogensis, wound healing, tissue engraftment and muscle tissue regeneration and/or tissue augmentation comprising administering a heparin-derivatized collagen matrix that includes at least one heparin-binding growth factor and/or heparin-binding adeno-associated virus (HB-AAV).

The heparin and HBGF/HB-AAV of the heparin-derivatized collagen matrix of the invention strongly associate via interactions with the heparin binding domains of the HBGFs and HB-AAVs and/or by ionic interactions of the growth factors/virus particles and heparin. This strong association results in slow desorption of the HBGFs and/or HB-AAVs from the collagen over time. Heparin may potentiate the biological activities of heparin-binding growth factors and may protect the HBGFs from proteolytic degradation. This interaction between heparin and the HBGFs and/or HB-AAVs results in a heparin-derivatized collagen matrix that retains the bioactive HBGFs and/or HB- AAVs at the injury site for a longer period of time.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a graph depicting the in vitro release of rhBMP-2 from various collagen scaffolds before and after heparin derivatization.

FIG. 2 is a graph depicting the in vitro release of adeno-associated virus type 2 (AAV2)-cytomegalovirus (CMV)—enhanced green fluorescent protein (EGFP) from a collagen scaffold before and after heparinization.

FIG. 3 is a bar graph depicting the in vitro release of AAV2 released from heparinized Scaffold 1 treated with either 5 or 20 mg/mL heparin fragments.

FIG. 4 is a bar graph depicting the heparin content results of collagen scaffolds using various concentrations of heparin fragments in the derivatization procedure.

FIG. 5 is a bar graph depicting the sulfur content results of collagen coated dextran particles treated with heparin fragments compared to untreated controls.

FIG. 6 is a bar graph depicting the results of the determination of 1× and 4× heparin fragment molecular weights using gel permeation chromatography (GPC).

### DETAILED DESCRIPTION OF THE INVENTION

A description of example embodiments of the invention follows.

The present invention relates to the need for stabilized and prolonged delivery of heparin-binding growth factors (HBGFs) and/or heparin-binding adeno-associated virus particles (HB-AAVs) to local sites of repair. More specifically, the present invention relates to a heparin-derivatized collagen matrix comprising a fragment of heparin covalently linked to a collagen scaffold, wherein the fragment of heparin has a molecular weight of less that about 15 kDa, and at least one heparin-binding growth factor (HBGF) and/or heparin-binding adeno-associated virus (HB-AAV).

Examples of the HBGFs of the present invention include, but are not limited to, a fibroblast growth factor (FGF),vascular endothelial growth factor (VEGF), heparin- binding epidermal growth factor (HBEGF), a member of the transforming growth factor-β (TGF-β) superfamily, keratinocyte growth factor (KGF), pleiotrophin, placental growth factor (PlGF), hepatocyte growth factor, interferon-gamma (IFN-gamma), platelet-derived growth factor (PDGF), interleukin-8 (IL-8), macrophage inflammatory protein-1 (MIP-1), interferon-gamma-inducible protein-10 (IP-10) or HIV-Tat transactivating factor. Many of these angiogenesis-related HBGFs are implicated in the stimulation of target cell proliferation, differentiation and organization in developing tissues.

In a preferred embodiment of the invention, the HBGF is a member of the TGF-β superfamily. In another preferred embodiment, the member of the TGF-β superfamily is bone morphogenetic protein 2 (BMP-2). Both the TGF-β superfamily and bone morphogenetic protein 2 are known to play a role in bone and cartilage regeneration and repair.

In one embodiment of the invention, the HB-AAV is selected from the group consisting of fibroblast growth factor (FGF), vascular endothelial growth factor (VEGF), heparin-binding epidermal growth factor (HBEGF), the transforming growth factor-b (TGF-β) superfamily, keratinocyte growth factor (KGF), pleiotrophin, placental growth factor (PlGF), hepatocyte growth factor, interferon-gamma (IFN-gamma), platelet-derived growth factor (PDGF), interleukin-8 (IL-8), macrophage inflammatory protein-1 (MIP-1), interferon-gamma-inducible protein-10 (IP-10) or HIV-Tat transactivating factor and combinations thereof. For example, HB-AAV is adeno- associated virus-2 (AAV-2) that promotes the expression of one of the members of the TGF-β superfamily. In another example, the HB-AAV is AAV-2 that promotes expression of bone morphogenetic protein 2. The AAV2 is used as a gene delivery agent to efficiently transfer genes of interest to cells to provide long-term protein expression.

In one embodiment of the invention, the heparin-derivatized collagen matrix comprises a fragment of heparin covalently linked to a collagen scaffold. The fragments of heparin are covalently attached to the collagen scaffold (such as, for example, a collagen sponge) by end-point attachment. The end-point attachment method is described, for example, in U.S. Pat. No. 4,613,665, issued to Larm, the entire contents of which is hereby incorporated by reference in its entirety. This end-point attachment method of attaching fragments of heparin to a collagen scaffold enables heparin loading levels (0.03-30% w/w) on the surfaces of the collagen scaffold. This in turn allows for the free movement of the heparin chains for efficient interaction with heparin binding domain of HBGFs and/or HB-AAVs. As described in greater detail below, the high heparin loading level translates into stabilized and prolonged delivery of HBGFs to local sites of repair.

Approaches to localize and prolong HBGF delivery at sites of injury include delivery from biodegradable PLGA scaffolds and microspheres, fibrin glue, injectable polymeric depots, self-assembling peptides, collagen fibrils, hyaluronan films, ethylene vinyl acetate copolymer implants, alginate hydrogels, drug delivery catheters, osmotic pumps and gene transfer methods. (See for example Lee, H., Cusick R A, Browne F, Ho Kim T, Ma P X, Utsunomiya H, Langer R, Vacanti J P. Transplantation. (2002) May 27; 73(10):1589-93; Cleland J L, Duenas E T, Park A, Daugherty A, Kahn J, Kowalski J, Cuthbertson A. J Control Release. 2001 May 14; 72(1-3):13-24; U.S. Pat. No. 6,197,325 “Supplemented and unsupplemented tissue sealants, methods of their production and use”; Patrick C. H. Hsieh, Michael E. Davis, Joseph Gannon, Catherine MacGillivray, and Richard T. Lee. J Clin Invest. 2006 Jan. 4; 116(1): 237-248;Bentz H, Schroeder J A, Estridge T D. J Biomed Mater Res. 1998 Mar. 15; 39(4):539-48; Peattie R A, Rieke E R, Hewett E M, Fisher R J, Shu X Z, Prestwich G D. Biomaterials. 2006 March; 27(9):1868-75; Wong W C, Yu Y, Wallace A L, Gianoutsos M P, Sonnabend D, Walsh W R. ANZJ. Surg. 2003 December; 73(12):1022-7; Kaftan H, Hosemann W, Junghans D, Gopferich A, Schindler E, Beule A. HNO. 2005 June; 53(6):539-42, 544-5; Van Belle E, Maillard L, Tio F O, Isner J M. Biochem Biophys Res Commun. 1997 Jun. 18; 235(2):311-6; Fowlkes J L, Thrailkill K M, Liu L, Wahl E C, Bunn R C, Cockrell G E, Perrien D S, Aronson J, Lumpkin C K Jr. J Bone Miner Res. 2006 September; 21(9):1359-66; Yukawa, H. et al., Gene Ther 2000 June; 7(11):942-949, the entire contents of each is hereby incorporated by reference in its entirety. See also, European Patent EP1446100 “Injectable depot compositions and uses thereof.”) These delivery vehicles may provide therapeutic concentrations of growth factors to local sites over time, however stabilization of the protein and amplification of the protein activity has been challenging with these approaches.

The fragments of heparin of the invention can be prepared by methods described more fully in Examples 1 and 2 of the present application. In one embodiment, heparin fragments should have a molecular weight of less than about 15 kDa. More particularly, the heparin fragments should have a molecular weight of between about 12 and about 13 kDa or of between about 5 and about 6 kDa can be prepared by nitrous acid degradation. The term “fragment” has been used in the singular but the plural form is also intended. In a preferred embodiment, the collagen scaffold will be optimally loaded with a concentration of heparin fragments that allows for efficient binding of an appropriate concentration of HBGFs or HB-AAVs. Collagen scaffolds can be prepared with up to 30% w/w heparin fragments. The heparin fragments prepared by this method would retain their antithrombin III activity, however they could be further purified by methods known in the art so that the fragments have little to no anticoagulant activity.

The collagen scaffold comprises, for example, a collagen film, sponge, solution, suspension or particle. Examples of collagen scaffolds include, but are not limited to, a collagen film or a collagen sponge such as a Helistat®, Integra Mozaik™, INTEGRA Bilayer Matrix Wound Dressing, NeuraGen®, NeuraWrap™, TenoGlide™, DuraGen®, DuraGen Plus®, BioMend®, CollaTape®, CollaCote®, CollaPlug® (all available from Integra LifeSciences Corporation, Plainsboro, N.J.), Avitene Sheets®, Ultrafoam™ (both available from Davol, Cranston, R.I.), Gelfoam® (Pfizer Inc., New York, N.Y.), Instat™ (Ethicon, Cincinnati, Ohio), a cross-linked Matricel sponge or a ACI-Maix™ collagen scaffold (both available from Matricel GmbH, Herzogenrath, Germany), a collagen solution, suspension, or particle such as Cosmoderm1®, Cosmoplast®, Zyderm1®, Zyderm2®, and Zyplast® (all available from Inamed, Santa Barbara, Calif.), Surgifoam™, Surgiflo™ (Ethicon, Cincinnati, Ohio), a fabrillar collagen such as Helitene® (Integra LifeSciences Corporation, Plainsboro, N.J.) and Avitene® Flour (Davol, Cranston, R.I.).

After the fragments of heparin are covalently linked to the collagen scaffold, the heparinized collagen scaffold can be purified by known methods. After purification, at least one HBGF and/or HB-AAV can be loaded onto the scaffold under the preferred conditions for the particular HBGF and/or HB-AAV.

Combinations of growth factors, AAVs and/or HB-AAV may also be loaded onto the scaffold to provide additive or synergistic activity. Combinations of HBGFs or HB-AAVs may include two or more of these species or combinations of HBGFs and HB-AAVs. Examples of growth factor combinations that could be used to promote an angiogenic response include (1) PDGF and FGF and (2) insulin-like growth factor -1 (IGF-1) and VEGF. Examples of growth factor combinations that could be used to promote a chondrogenic response include (1) TGF-β and BMP-2 and (2) TGF-β and IGF-1. Examples of growth factor combinations that could be used to promote osteogenesis include (1) IGF-1 and PDGF, (2) IGF-1 and TGF-β-1 and (3) IGF-1 and FGF. Similarly, AAV2 constructs that transduce cells for therapeutic protein expression may be used in combination with either recombinant HBGFs or other AAV2 species to provide additive or synergistic activity. It may be desirable to combine an HBGF with a non-HBGF to provide rapid release of the non-HBGF with a more sustained delivery of the HBGF. It may also be desirable to combine an HBGF with an HB-AAV for a sustained delivery of the HBGF with long-term gene expression provided by the HB-AAV. Methods for loading HBGFs and HB-AAVs onto heparin-derivatized collagen matrices are described, for example, in Examples 6 and 7.

As illustrated in the Examples, loading a heparin-binding growth factor (HBGF) onto the heparin-derivatized collagen matrix of the present invention results in a decrease in the delivery rate of the HBGF. For example, heparin modified scaffolds showed a dramatic decrease in elution of BMP-2 compared to unmodified collagen scaffolds. In a study comparing two unmodified scaffolds and two heparin modified collagen scaffolds, unmodified scaffolds eluted 77% and 23%, respectively, after 14 days. Collagen scaffolds modified by heparin fragments having a molecular weight between 12 and 13 kDa, eluted 16% and 4%, respectively, after 14 days. Similarly, as described in Example 7, loading an HB-AAV onto a derivatized collagen matrix of the present invention results in a decrease in the delivery rate of the HB-AAV. For example, heparin modified scaffolds treated with either 5 mg/mL or 20 mg/mL of heparin fragments showed 16.9% and 8.75% capsids released, respectively, after 4 hours.

The invention also relates to methods for promoting bone growth, bone repair, bone development, cartilage repair, neo-angiogenesis, wound healing, tissue engraftment and muscle tissue regeneration and/or tissue augmentation comprising administering a heparin-derivatized collagen matrix that includes at least one heparin-binding growth factor (HBGF) and/or at least one heparin-binding AAV (HB-AAV).

The heparin-derivatized collagen matrix of the present invention can be administered to promote bone growth, bone repair, cartilage repair, bone development, neo-angiogensis, wound healing, tissue engraftment and muscle tissue regeneration and/or tissue augmentation via a variety of delivery routes, such as transdermal, ophthalmic, nasal, pulmonary, injectable or implantable delivery routes. The heparin-derivatized collagen matrix can also take a variety of delivery forms including, for example, patches, rods, suspensions, solutions and dry particulates. The heparin-derivatized collagen matrix can also be bioresorbable (biodegradable) or biostable (non-resorbable).

The invention also includes a method for promoting bone growth, bone repair, bone development and/or cartilage repair by administering a heparin-derivatized collagen matrix comprising a heparin-derivatized collagen matrix comprising a fragment of heparin covalently linked to a collagen scaffold, wherein the fragment of heparin has a molecular weight of less that about 15 kDa, and at least one heparin-binding growth factor (HBGF) or heparin-binding adeno-associated virus (HB-AAVs) or combination thereof. In a preferred embodiment, the HBGF is a member of the TGF-β superfamily (e.g., BMP-2). In another preferred embodiment, the HBGF is IGF-1, VEGF or PDGF. In yet another embodiment, the HB-AAV is AAV-2 (e.g., AAV-2 for expression of a member of the TGF-β superfamily, IGF-1, FGF, VEGF or PDGF).

In one embodiment, the heparin-derivatized collagen matrix is loaded with an HBGF (e.g., a member of the TGF-β superfamily, IGF-1, FGF, VEGF or PDGF) and/or a heparin-binding adeno-associated virus (e.g., AAV2 for expression of a member of the TGF-β superfamily, IGF-1, FGF, VEGF or PDGF) or combination thereof with or without cells and implanted at a non-union boney defect to induce or accelerate bone repair.

In another embodiment, the heparin-derivatized collagen matrix is loaded with an HBGF (e.g., a member of the TGF-(superfamily, IGF-1, FGF, VEGF or PDGF) and/or a heparin-binding adeno-associated virus (e.g., AAV2 for expression of a member of the TGF-β superfamily, IGF-1, FGF, VEGF or PDGF) with or without cells and injected into a hairline fracture to accelerate bone repair.

In yet another embodiment, the heparin-derivatized collagen matrix is loaded with an HBGF (e.g., a member of the TGF-β superfamily, IGF-1, FGF, VEGF or PDGF) and/or a heparin-binding adeno-associated virus (e.g., AAV2 for expression of a member of the TGF-β superfamily, IGF-1, FGF, VEGF or PDGF) with or without cells and implanted at a chondral or osteochondral defect to induce or accelerate cartilage repair.

The invention also includes a method for promoting neo-angiogenesis by administering a heparin-derivatized collagen matrix comprising a heparin-derivatized collagen matrix comprising a fragment of heparin covalently linked to a collagen scaffold, wherein the fragment of heparin has a molecular weight of less that about 15 kDa, and at least heparin-binding growth factor (HBGF) and/or at least one heparin-binding AAV (HB-AAV). In a preferred embodiment, the HBGF is IGF, VEGF, FGF, PDGF, or a member of the TGF-β superfamily and/or a heparin-binding adeno-associated virus (e.g., AAV2 for expression of IGF, VEGF, FGF, PDGF or a member of the TGF-β superfamily).

In one embodiment, the heparin-derivatized collagen matrix is loaded with an HBGF (e.g., a member of the TGF-β superfamily, IGF-1, FGF, VEGF or PDGF) and/or a heparin-binding adeno-associated virus (e.g., AAV2 for expression of a member of the TGF-β superfamily, IGF-1, FGF, VEGF or PDGF) with or without cells and injected into ischemic tissue (e.g., infarcted myocardium, ischemic latissimus dorsi muscle flaps for dynamic cardiomyoplasty, peripheral artery disease) to create functional vasculature that may amplify the recovery of tissue ischemia.

In another embodiment, the heparin-derivatized collagen matrix is a patch and is loaded with an HBGF (e.g., a member of the TGF-β superfamily, IGF-1, FGF, VEGF or PDGF) and/or a heparin-binding adeno-associated virus (e.g., AAV2 for expression of a member of the TGF-β superfamily, IGF-1, FGF, VEGF or PDGF) with or without cells and sutured onto ischemic myocardium to induce the formation of new blood vessels under the patch.

In yet another embodiment, the heparin-derivatized collagen matrix comprises implantable electrodes or sensors are loaded with HBGF (e.g., a member of the TGF-β superfamily, IGF-1, FGF, VEGF or PDGF) and/or a heparin-binding adeno-associated virus (e.g., AAV2 for expression of a member of the TGF-β superfamily, IGF-1, FGF, VEGF or PDGF) to improve neo-angiogenesis in the surrounding fibrotic capsule to enhance the function of an implanted device.

The invention also includes a method for promoting wound healing by administering a heparin-derivatized collagen matrix comprising a heparin-derivatized collagen matrix comprising a fragment of heparin covalently linked to a collagen scaffold, wherein the fragment of heparin has a molecular weight of less that about 15 kDa, and at least heparin-binding growth factor (HBGF) and/or at least one heparin-binding AAV (HB-AAV). In a preferred embodiment, the HBGF is KGF, EGF, a member of the TGF-β superfamily, IGF-1, FGF, VEGF or PDGF and/or a heparin- binding adeno-associated virus (e.g., AAV2 for expression of KGF, EGF, a member of the TGF-β superfamily, IGF-1, FGF, VEGF or PDGF).

In one embodiment, the heparin-derivatized collagen matrix is in the form of a gel or patch loaded with an HBGF is KGF, EGF, or a member of the TGF-β superfamily, IGF-1, FGF, VEGF or PDGF and/or a heparin-binding adeno-associated virus (e.g., AAV2 for expression of a KGF, EGF, a member of the TGF-β superfamily, IGF-1, FGF, VEGF or PDGF) with or without cells and that is applied topically to a skin wound or burn to stimulate the wound healing response.

In another embodiment, the heparin-derivatized collagen matrix is in the form of a gel or patch loaded with an HBGF (e.g., KGF, EGF, a member of the TGF-β superfamily, IGF-1, FGF, VEGF or PDGF) and/or a heparin-binding adeno-associated virus (e.g., AAV2 for expression of KGF, EGF, a member of the TGF-β superfamily, IGF-1, FGF, VEGF or PDGF)with or without cells that applied topically to a perforation in a tympanic membrane to enhance healing of the membrane.

The invention also includes a method for promoting tissue engraftment and muscle tissue regeneration by administering a heparin-derivatized collagen matrix comprising a heparin-derivatized collagen matrix comprising a fragment of heparin covalently linked to a collagen scaffold, wherein the fragment of heparin has a molecular weight of less that about 15 kDa, and at least heparin-binding growth factor (HBGF) and/or at least one heparin-binding AAV (HB-AAV). In a preferred embodiment, the HBGF is HGF or a member of the TGF-β superfamily, IGF-1, FGF, VEGF or PDGF and the HB-AAV promotes the expression of HGF or a member of the TGF-β superfamily, IGF-1, FGF, VEGF or PDGF.

In one embodiment, the heparin-derivatized collagen matrix is loaded with HGF or FGF and/or a heparin-binding adeno-associated virus that promotes the expression of HGF or FGF and then seeded with myoblasts to improve the long-term survival and migration of these cells for regeneration of muscle tissue.

In another embodiment, the heparin-derivatized collagen matrix is loaded with VEGF or FGF and/or a heparin-binding adeno-associated virus that promotes the expression of VEGF or FGF and then seeded with hepatocytes to enhance scaffold vascularization for improved survival of transplanted hepatocytes. Therapeutic applications include treatment for end-stage liver disease and enzyme deficiencies.

In yet another embodiment, the heparin-derivatized collagen matrix in the form of a gel is loaded with IGF-1 and/or a heparin-binding adeno-associated virus that promotes the expression of IGF-1 and locally administered to a cross-facial nerve graft to enhance reinnervation of the orbicularis oculi muscle for treatment of facial paralysis.

The invention also includes a method for promoting tissue augmentation (e.g., dermal filler applications) by administering a heparin-derivatized collagen matrix comprising a heparin-derivatized collagen matrix comprising a fragment of heparin covalently linked to a collagen scaffold, wherein the fragment of heparin has a molecular weight of less that about 15 kDa, and at least heparin-binding growth factor (HBGF) and/or at least one heparin-binding AAV (HB-AAV). In a preferred embodiment, the HBGF is IGF-1 and the HB-AAV promotes the expression of IGF-1.

The invention also relates to the use of a heparin-derivatized collagen matrix to sequester adeno-associated virus type 2 (AAV-2) for virus-mediated transfection of cells. In one embodiment, AAV2-EGFP is loaded onto the heparin-derivatized collagen matrix.

In one embodiment, the heparin-derivatized collagen matrix is in the form of particles and is loaded with a HBGF (e.g., IGF-1) and/or a HB-AAV (e.g., AAV2 for expression of IGF-1) and insulin and injected into inguinal adipofascial flaps to increase the number of mature adipocytes as a method of adipofascial flap augmentation.

### EXAMPLES

### Example 1

### Preparation of 12-13 kDa Heparin Fragments (1× Fragments)

Porcine mucosal heparin (1 g) was dissolved in 300 mL deionized water and cooled to 0° C. under constant stirring. Sodium nitrite (10 mg) was added to the heparin sulfate solution as a concentrated aqueous solution (100 mg/mL sodium nitrite in deionized water). Acetic acid (2 mL) was added dropwise and the solution was left to stir at 0° C. for 2 hours. The heparin solution was then dialyzed twice against 4 L of saline for a total of 24 hours and twice against 4 L of deionized water for a total of 24 hours. The heparin solution was lyophilized.

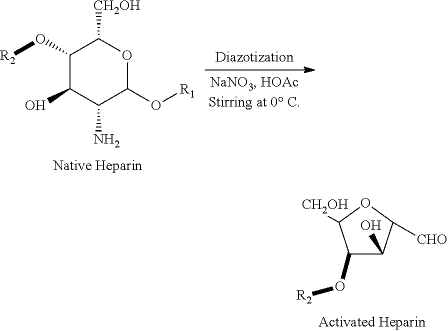

### Example 2

### Preparation of 5-6 kDa Heparin Fragments (4× Fragments)

Porcine mucosal heparin (1 g) was dissolved in 300 mL deionized water and cooled to 0° C. under constant stirring. Sodium nitrite (40 mg) was added to the heparin sulfate solution as a concentrated aqueous solution (100 mg/mL sodium nitrite in deionized water). Acetic acid (8 mL) was added dropwise and the solution was left to stir at 0° C. for 2 hours. The heparin solution was then dialyzed twice against 4 L of saline for a total of 24 hours and twice against 4 L of deionized water for a total of 24 hours. The heparin solution was lyophilized.

### Example 3

### Determination of 1× and 4× Heparin Fragment Molecular Weights Using Gel Permeation Chromatography (GPC)

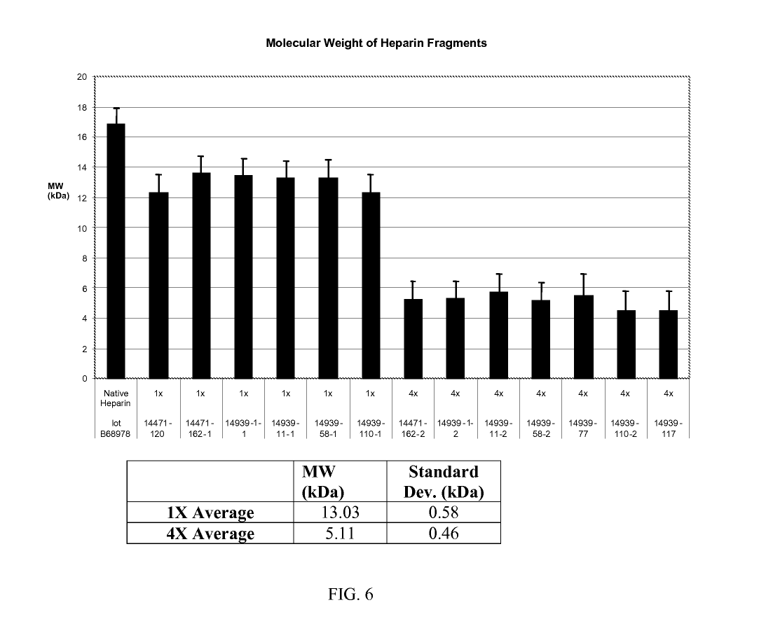

TriSEC 302 (Viscotek): This GPC system consists of an HPLC pump (Viscotek VE1121), solvent degasser (Viscotek VE 7510), one column (Viscotek ViscoGEL GMPWXL) and four detectors in tandem: light scattering (RALLS—Right Angle Laser Light Scattering and LALLS—Low Angle Laser Light Scattering), refractive index and viscometer (Viscotek TDA model 302 with LALLS). Sample injections were performed by autosampler (Viscotek VE 5200) with a 100 μL injection volume. The mobile phase was an aqueous solution of 0.15M sodium nitrate at pH 7 and a flow rate of 0.5 mL/min and 30° C. column temperature. Data from light scattering, viscometer and refractive index detectors were collected and processed with OmniSEC 3.0 software (Viscotek) to give the weight-average molecular weight (Mw), number-average molecular weight (Mn), polydispersity (Mw/Mn) and intrinsic viscosity (IV) (See FIG. 6)

In the following examples, two collagen scaffolds were used. The two scaffolds differed in their physical appearance, collagen type, thickness and density and are defined in Table 1.

TABLE 1
Scaffold properties

| | Scaffold 1 | Scaffold 2 |
|---|---|---|
| Physical Appearance | porous, spongelike | dense, fibrous |
| Collagen Type | bovine type I | porcine type I/III |
| | (flexor tendon) | (peritoneum) |
| Thickness (mm) | 5 | 0.3 |
| Density (mg/cm³) | 12 | 471 |
| Density (normalized | 1 | 39 |
| to Scaffold 1) | | |

### Example 4

### Derivatization of Heparin Fragments onto Collagen Scaffolds through the Formation of a Schiff Base and Reductive Amination

A 20 mg/mL heparin fragment solution was prepared by dissolving heparin fragments in phosphate buffered saline (PBS) pH 7.2. This solution was added at a ratio of the surface area of the collagen scaffold (top face) to volume at 1.9 mL/cm2 collagen. For example, 3 mm and 6 mm biopsy punches of a collagen scaffold have top face surface areas of 0.07 cm2 and 0.28 cm2, respectively. Sodium cyanoborohydride solution was prepared at 40 mg/mL in PBS. This solution was added to the collagen scaffolds in heparin fragment solution at a volume of 50.5 μl/cm2 collagen. The scaffolds were left to react at room temperature for 24 hours. The scaffolds were rinsed with excess PBS for 24 hours and excess deionized water for 24 hours. Following the washes the scaffolds were lyophilized.

### Example 5

### Quantifying Covalently Linked Heparin on Collagen Scaffolds Using Elemental Analysis

6 mm diameter discs of two different collagen scaffolds (Scaffold 1 and Scaffold 2) were derivatized with both 1× and 4× heparin fragments. Controls were prepared using washed scaffolds, scaffolds incubated with native heparin without a reducing agent as well as scaffolds incubated with heparin fragments also without the reducing agent (sodium cyanoborohydride). The scaffolds were analyzed for sulfur content (w/w %) using atomic absorption spectroscopy. The sulfur content of heparin used was known (11.11% w/w) and allowed calculation of the amount of heparin present (w/w %) on each of the scaffolds. The control groups showed no significant amount of heparin bound to the scaffolds. Scaffold 1 and Scaffold 2 showed 5.58 and 18.09% heparin (w/w) respectively when derivatized with 1× fragments and 7.20 and 22.14% heparin (w/w) when derivatized with 4× fragments. (See Table 2, below).

TABLE 2
% Heparin (w/w)

| | Native | 1X Hep | 4X Hep | | |
|---|---|---|---|---|---|
| | Heparin (no | Frag (no | Frag (no | 1X Hep | 4X Hep |
| Scaffold | NaCNBH₃) | NaCNBH₃) | NaCNBH₃) | Frag | Frag |
| Scaffold 1 | 0.09 | 0.09 | 0.00 | 5.58 | 7.20 |
| Scaffold 2 | 0.81 | 0.99 | 0.27 | 18.09 | 22.14 |

### Example 6

### In Vitro Release of rhBMP-2 from Heparin-Derivatized Collagen Matrix

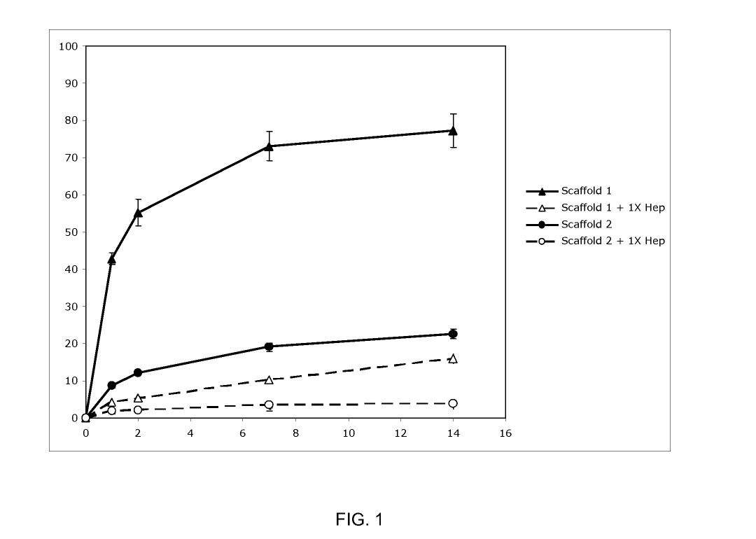

3 mm diameter punches of 1× heparinized Scaffold 1 and Scaffold 2 were prepared. These heparinized scaffolds as well as non-heparinized controls were each loaded with 5 μg rhBMP-2 in 30% ethanol, 0.01% triflouroacetic acid and lyophilized (n=3/group). Each scaffold was placed in a separate 1.7 mL Eppendorf tube and 1 mL release media (PBS+1% HSA; pH 7.4) was added. Samples were incubated at 37° C. under constant shaking. At 2 hours, 1, 2, 4, 7, 10, and14 days, 100 μl release media was removed from each tube and replaced with fresh release media. Media was analyzed by ELISA for BMP-2 over a 14 day period. Heparin modified scaffolds showed a dramatic decrease in elution of BMP-2. Untreated scaffolds eluted 77% and 23%, respectively, for Scaffold 1 and Scaffold 2 after 14 days. 1× heparinized scaffolds eluted 16% and 4%, respectively, for Scaffold 1 and Scaffold 2 after 14 days. (FIG. 1)

### Example 7

### In Vitro Release of AAV2 from Heparin-Derivatized Collagen Matrix

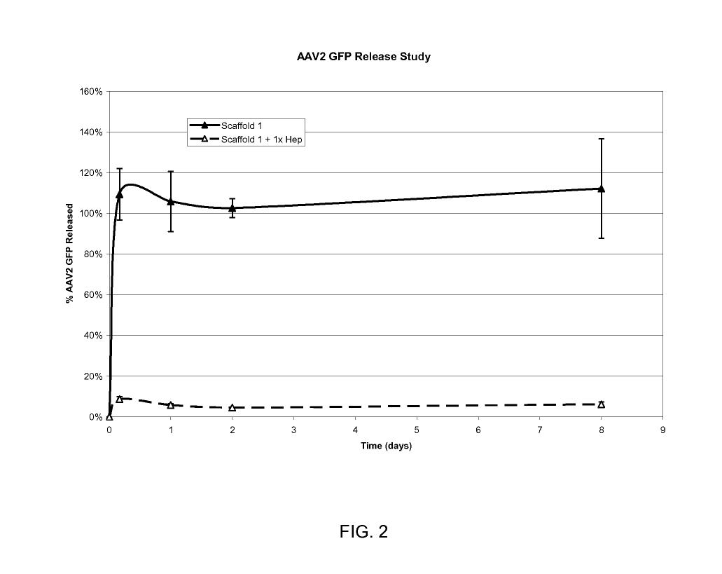

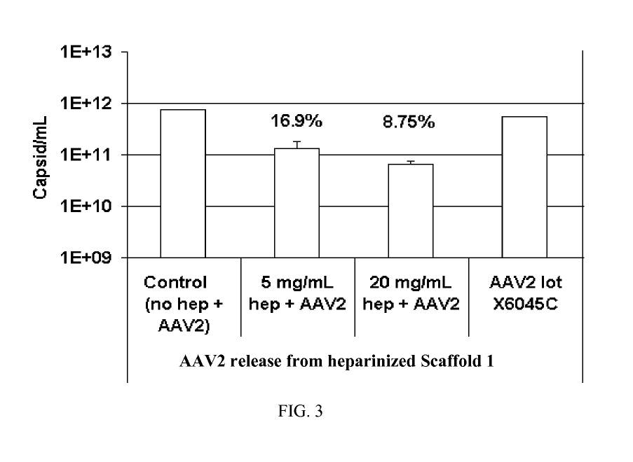

6 mm diameter punches of 1× heparinized Scaffold 1 were prepared using 20 mg/mL heparin fragments. These heparinized scaffolds as well as non-heparinized controls were each loaded with 1.58e11DRP/scaffold with AAV2 CMV EGFP and lyophilized (n=3/group). Each scaffold was placed in a separate 1.7 mL eppendorf tube. AAV2 control (1.58e11DRP) was pipetted into 1.7 mL eppendorf tube. One mL of release medium (DME; 10% FBS; 1× Pen. Strep.) was added to each eppendorf tube. Samples were incubated at 37° C. on a 360° rotating platform for 8 days. At 4 hours, 1, 2, and 8 days, 100 μl release media was removed from each tube. Media was analyzed by capsid ELISA and for infectivity on 293 cells. For untreated scaffolds, 100% AAV2 was released at 4 hours which remained constant over 8 days compared to controls. 1× heparin treated scaffolds released 9% AAV2 after 4 hours with no additional release over 8 days compared to controls (FIG. 2). The AAV2 released from both scaffolds was active for up to 8 days. In a related experiment, 1× heparinized Scaffold 1 samples were prepared using 5 and 20 mg/mL heparin fragment solutions and showed 16.9% and 8.75% release of the initially loaded AAV2 over the same time period, respectively (FIG. 3). These results suggest the ability to modulate the elution profile by pretreatment with various concentrations of heparin fragment solutions.

### Example 8

### Derivatization of Various Concentrations of Heparin Fragments onto Collagen Scaffolds through the Formation of a Schiff Base and Reductive Amination

20 mg/mL, 15 mg/mL, 10 mg/mL, 5 mg/mL, and 0 mg/mL heparin fragment solutions were prepared by dissolving heparin fragments in phosphate buffered saline (PBS) pH 7.2. Solutions were added to the collagen scaffolds at a volume of 1.9 mL/cm² collagen. Sodium cyanoborohydride solution was prepared at 40 mg/mL in PBS. This solution was added to the collagen scaffolds in heparin fragment solution at a volume of 50.5 μl/cm² collagen. The scaffolds were left to react at room temperature for 24 hours. The scaffolds were rinsed with excess PBS for 24 hours and excess deionized water for 24 hours. Following the washes the scaffolds were lyophilized.

### Example 9

### Quantifying Covalently Linked Heparin on Collagen Scaffolds Treated with Various Concentrations of Heparin Fragments

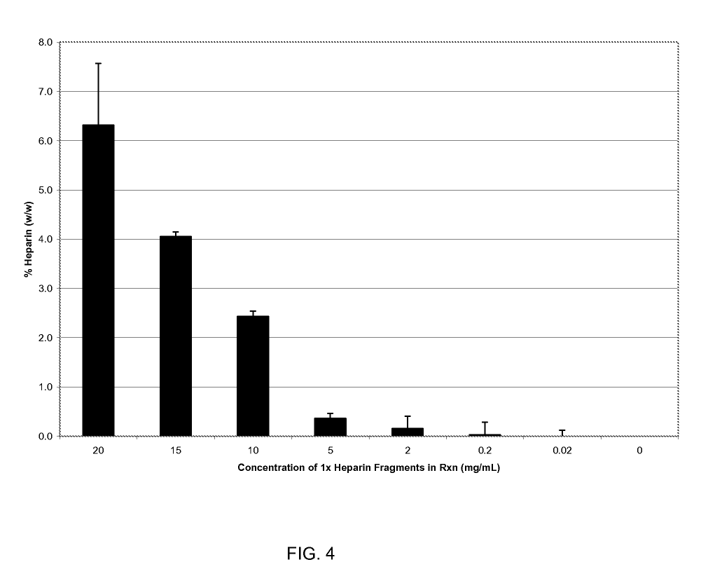

6 mm diameter discs of Scaffold 1 were derivatized with 20 mg/mL, 15 mg/mL, 10 mg/mL, 5 mg/mL, 2 mg/mL, 0.2 mg/mL, 0.02 mg/mL and 0 mg/mL 1× heparin fragment solutions. The scaffolds were analyzed for sulfur content (w/w %) using atomic absorption spectroscopy. The sulfur content of heparin used was known (11.11% w/w) and allowed us to calculate the amount of heparin present (w/w %) on each of the scaffolds. The control group (0 mg/mL) showed no significant amount of heparin bound to the scaffolds. Scaffolds contained 6.32, 4.05, 2.43, 0.36, 0.15, 0.03, and 0.00% heparin (w/w), respectively, when derivatized with 20 mg/mL, 15 mg/mL, 10 mg/mL, 5 mg/mL, 2 mg/mL, 0.2 mg/mL and 0.02 mg/mL 1× fragments (FIG. 4).

### Example 10

### Derivatization of Heparin Fragments onto Collagen Particles through the Formation of a Schiff Base and Reductive Amination

A 40 mg/mL heparin fragment solution was prepared by dissolving heparin fragments in phosphate buffered saline (PBS) pH 7.2. 10 mL of the 40 mg/mL heparin fragment solution was added to 1 g collagen coated dextran particles in 10 mL PBS (20 mg/mL final heparin fragment concentration). Sodium cyanoborohydride solution was prepared at 40 mg/mL in PBS and 530 μl was added to the collagen particle suspension in heparin fragment solution. The particles were left to react at room temperature for 24 hours. The particles were rinsed with excess PBS for 24 hours and excess deionized water for 24 hours. Following the washes the particles were lyophilized.

### Example 11

### Quantifying Covalently Linked Heparin on Collagen Particles Using Elemental Analysis

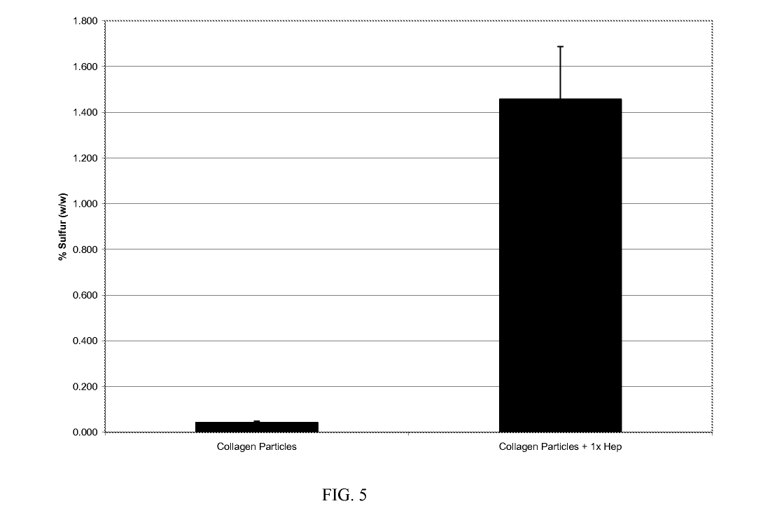

Collagen coated dextran particles were derivatized with 1× heparin fragments. Controls were prepared using washed particles. The particles were analyzed for sulfur content (w/w %) using atomic absorption spectroscopy. The control particles contained 0.04% (w/w) sulfur compared to 1× derivatized particles which contained 1.46% (w/w) sulfur (FIG. 5). This difference in sulfur content calculates to 12.70% (w/w) heparin modification of the surface of the 1× derivatized collagen coated dextran particles.

While this invention has been particularly shown and described with references to example embodiments thereof, it will be understood by those skilled in the art that various changes in form and details may be made therein without departing from the scope of the invention encompassed by the appended claims.

1. A heparin-derivatized collagen matrix comprising a fragment of heparin covalently linked to a collagen scaffold, wherein the fragment of heparin has molecular weight of less than about 15 kDa, and at least one heparin-binding growth factor (HBGF) or heparin-binding adeno-associated virus (HB-AAV) or a combination thereof.

2. The heparin-derivatized collagen matrix of claim 1, wherein the HBGF is selected from the group consisting of fibroblast growth factor (FGF), vascular endothelial growth factor (VEGF), heparin-binding epidermal growth factor (HBEGF), the transforming growth factor-β (TGF-β) superfamily, keratinocyte growth factor (KGF), pleiotrophin, placental growth factor (PlGF), hepatocyte growth factor, interferon-gamma (IFN-gamma), platelet-derived growth factor (PDGF), interleukin-8 (IL-8), macrophage inflammatory protein-1 (MIP-1), interferon-gamma-inducible protein-10 (IP-10) or HIV-Tat transactivating factor and combinations thereof.

3. The heparin-derivatized collagen matrix of claim 2, wherein the HBGF is at least one member of the TGF-β superfamily.

4. The heparin-derivatized collagen matrix of claim 3, wherein the HBGF is bone morphogenetic protein 2 (BMP-2).

5. The heparin-derivatized collagen matrix of claim 1, wherein the HB-AAV is selected from the group consisting of AAVs that promote the expression of fibroblast growth factor (FGF), vascular endothelial growth factor (VEGF), heparin-binding epidermal growth factor (HBEGF), the transforming growth factor-b (TGF-β) superfamily, keratinocyte growth factor (KGF), pleiotrophin, placental growth factor (PlGF), hepatocyte growth factor, interferon-gamma (IFN-gamma), platelet-derived growth factor (PDGF), interleukin-8 (IL-8), macrophage inflammatory protein-1 (MIP- 1), interferon-gamma-inducible protein-10 (IP-10) or HIV-Tat transactivating factor and combinations thereof.

6. The heparin-derivatized collagen matrix of claim 5, wherein HB-AAV is adeno-associated virus-2 (AAV-2) that promotes the expression of one of the members of the TGF-β superfamily.

7. The heparin-derivatized collagen matrix of claim 6, wherein the HB-AAV is adeno-associated virus-2 (AAV-2) that promotes the expression of bone morphogenetic protein 2 (BMP-2).

8. The heparin-derivatized collagen matrix of claim 1, wherein the fragment of heparin has a molecular weight less than about 15 kDa.

9. The heparin-derivatized collagen matrix of claim 8, wherein the fragment of heparin has a molecular weight between about 12 kDa and 13 kDa.

10. The heparin-derivatized collagen matrix of claim 8, wherein the fragment of heparin has a molecular weight between about 5 kDa and 6 kDa.

11. The heparin-derivatized collagen matrix of claim 1, wherein the HBGF or HB-AAV or a combination thereof promotes bone growth, bone repair and/or bone development.

12. The heparin-derivatized collagen matrix of claim 1, wherein the HBGF or HB-AAV or a combination thereof promotes cartilage repair.

13. A method of promoting bone or cartilage growth, repair and/or development comprising administering a heparin-derivatized collagen matrix comprising a fragment of heparin covalently linked to a collagen scaffold, wherein the fragment of heparin has a molecular weight of less than about 15 kDa, and at least one heparin-binding growth factor (HBGF) or heparin-binding adeno-associated virus (HB-AAV) or a combination thereof.

14. (canceled)

15. A method of promoting neo-angiogenesis comprising administering the heparin-derivatized collagen matrix of claim 1.

16. A method of promoting tissue regeneration or augmentation comprising administering a heparin-derivatized collagen matrix comprising a fragment of heparin covalently linked to a collagen scaffold, wherein the fragment of heparin has a molecular weight of less than about 15 kDa, and at least one heparin-binding growth factor (HBGF) or heparin-binding adeno-associated virus (HB-AAV) or a combination thereof.

17. (canceled)
